(19) United States
(12) Patent Application Publication
(10) Pub. No.: US 2025/0022607 A1
(43) Pub. Date: Jan. 16, 2025

(54) SYSTEM AND METHODS FOR QUANTIFYING AN IMPACT OF STORMWATER BEST MANAGEMENT PRACTICES

(71) Applicant: The Florida State University Research Foundation, Inc., Tallahassee, FL (US)

(72) Inventor: Nasrin Alamdari, Tallahassee, FL (US)

(21) Appl. No.: 18/772,682

(22) Filed: Jul. 15, 2024

Related U.S. Application Data
(60) Provisional application No. 63/513,454, filed on Jul. 13, 2023.

(51) Int. Cl. G16H 50/30

(52) U.S. Cl. CPC G16H 50/30

(57) ABSTRACT

Disclosed herein are a system and methods for determining an area's vulnerability to waterborne diseases and evaluating stormwater best management practices (BMPs) to mitigate vulnerabilities, according to various implementations. Generally, the disclosed system is configured to obtain data relating to a number of waterborne disease cases in a given area and corresponding socioeconomic data for the area. Based on these datasets, the disclosed system can determine a vulnerability index that is indicative of the area's vulnerability to waterborne diseases. The system can then predict how various stormwater BMPs can impact the vulnerability index (e.g., positively or negatively) to evaluate how implementing stormwater BMPs can improve the subject area's vulnerability to waterborne diseases. In some cases, the system and methods described herein may be implemented via a software tool; however, other implementations are contemplated herein.

### CROSS-REFERENCE TO RELATED APPLICATIONS

This application claims the benefit of priority to U.S. Provisional Patent Application No. 63/513,454, filed on Jul. 13, 2023, entitled “SYSTEM AND METHODS FOR QUANTIFYING AN IMPACT OF STORMWATER BEST MANAGEMENT PRACTICES,” the contents of which are hereby incorporated by reference in their entirety.

### BACKGROUND

There has been a growing concern about waterborne disease prevalence, especially in underserved communities. Stormwater best management practices (BMPs) are engineered practices that can restore the predevelopment hydrologic cycle through physical, chemical, and biological processes, thereby controlling surface runoff, and improving water quality while providing social, ecological, economic, and health benefits to communities. Although stormwater BMPs have been proven to effectively impact runoff quantity and quality, their effectiveness on waterborne disease reduction has not been investigated.

### SUMMARY

Disclosed herein are a system and methods for determining an area's vulnerability to waterborne diseases and evaluating stormwater best management practices (BMPs) to mitigate vulnerabilities. Generally, the disclosed system is configured to obtain data relating to a number of waterborne disease cases in a given area and corresponding socioeconomic data for the area. Based on these datasets, the disclosed system can determine a vulnerability index that is indicative of the area's vulnerability to waterborne diseases. The system can then predict how various stormwater BMPs can impact the vulnerability index (e.g., positively or negatively) to evaluate how implementing stormwater BMPs can improve the subject area's vulnerability to waterborne diseases. In some cases, the system and methods described herein may be implemented via a software tool; however, other implementations are contemplated herein.

In some aspects, the techniques described herein relate to a computer implemented method for determining a vulnerability index score including: obtaining, by one or more computing devices, a first data set including waterborne disease case data for a first geographical area; obtaining, by the one or more computing devices, a second data set including socioeconomic data for the first geographical area; determining, by the one or more computing devices, the vulnerability index score for the first geographical area based on the first data set and the second data set; and presenting, by the one or more computing devices, a first graphical user interface that indicates the vulnerability index score.

In some aspects, the techniques described herein relate to a method, further including: simulating, by the one or more computing devices, an implementation of one or more stormwater best management practices (BMPs) on the first geographical area; and predicting, by the one or more computing devices, an impact on the vulnerability index score due to the one or more stormwater BMPs based on the simulation.

In some aspects, the techniques described herein relate to a method, wherein the impact of the one or more stormwater BMPs on the vulnerability index score is simulated using a machine learning model.

In some aspects, the techniques described herein relate to a method, further including: presenting, by the one or more computing devices, a second graphical interface that indicates the one or more stormwater BMPs and the predicted impact on the vulnerability index score due to the one or more stormwater BMPs.

In some aspects, the techniques described herein relate to a method, further including: updating, by the one or more computing devices, the first graphical interface to include an indication of the one or more stormwater BMPs and the predicted impact on the vulnerability index score due to the one or more stormwater BMPs.

In some aspects, the techniques described herein relate to a method, wherein the first geographical area is a country, a state, a county, or a city.

In some aspects, the techniques described herein relate to a method, wherein the vulnerability index score is determined using a predictive model.

In some aspects, the techniques described herein relate to a method, wherein the predictive model is a machine learning model.

In some aspects, the techniques described herein relate to a method, wherein the first graphical user interface includes a map of at least the first geographical area.

In some aspects, the techniques described herein relate to a method, wherein the map visually indicates the vulnerability index score.

In some aspects, the techniques described herein relate to a system for determining a vulnerability index score including: at least one computing device; a computer-readable medium with computer-executable instructions stored thereon that when executed by the at least one computing device cause the at least one computing device to: obtain a first data set including waterborne disease case data for a first geographical area; obtain a second data set including socioeconomic data for the first geographical area; determine the vulnerability index score for the first geographical area based on the first data set and the second data set; and present a first graphical user interface that indicates the vulnerability index score.

In some aspects, the techniques described herein relate to a system, further including computer-executable instructions stored thereon that when executed by the at least one computing device cause the at least one computing device to: simulate an implementation of one or more stormwater best management practices (BMPs) on the first geographical area; and predict an impact on the vulnerability index score due to the one or more stormwater BMPs based on the simulation.

In some aspects, the techniques described herein relate to a system, wherein the impact of the one or more stormwater BMPs on the vulnerability index score is simulated using a machine learning model.

In some aspects, the techniques described herein relate to a system, further including computer-executable instructions stored thereon that when executed by the at least one computing device cause the at least one computing device to: present a second graphical interface that indicates the one or more stormwater BMPs and the predicted impact on the vulnerability index score due to the one or more stormwater BMPs.

In some aspects, the techniques described herein relate to a system, further including computer-executable instructions stored thereon that when executed by the at least one computing device cause the at least one computing device to: update the first graphical interface to include an indication of the one or more stormwater BMPs and the predicted impact on the vulnerability index score due to the one or more stormwater BMPs.

In some aspects, the techniques described herein relate to a system, wherein the first geographical area is a country, a state, a county, or a city.

In some aspects, the techniques described herein relate to a system, wherein the vulnerability index score is determined using a predictive model.

In some aspects, the techniques described herein relate to a system, wherein the predictive model is a machine learning model.

In some aspects, the techniques described herein relate to a system, wherein the first graphical user interface includes a map of at least the first geographical area.

In some aspects, the techniques described herein relate to system, wherein the map visually indicates the vulnerability index score.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

FIG. 1 is a block diagram of a system for determining an area's vulnerability to waterborne diseases and evaluating stormwater BMPs to mitigate vulnerabilities, according to some implementations;

FIG. 2 is an illustration of one example of how the vulnerability evaluator may calculate the vulnerability index score for a geographical area; and

FIG. 3 is an illustration of a method for predicting an impact of one or more stormwater BMPs on a vulnerability index score for a geographical area.

Various objects, aspects, and features of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Waterborne disease transmitted by microbiologically contaminated water can cause a variety of illnesses including neurological, respiratory, and gastrointestinal illnesses, bloodstream infections, and skin problems. According to the Centers for Disease Control and Prevention's (CDC) latest report on the burden of waterborne diseases in the United States, 7.15 million illnesses were caused by waterborne diseases, leading to 6,630 deaths, with the direct cost of $3.3 billion on the health care system in 2015. In addition, 82 waterborne disease outbreaks were reported in 2015 across the U.S., where the highest number of outbreaks were linked to recreational water exposure (57%). However, drinking water exposure was the leading cause of death, responsible for 25 of the 27 deaths in total. Florida experienced the highest frequency of waterborne disease outbreaks in the U.S., with a total number of 15 outbreaks. Most Common waterborne diseases in the U.S. included otitis externa (swimmer's ear), norovirus infection, giardiasis, and cryptosporidiosis, which accounted for 65%, 19%, and 6% of the total illnesses, respectively.

One of the main causes of the large waterborne outbreak has been Escherichia coli (E. coli). In two outbreaks of waterborne diseases, 45% and 35% of patients' stool samples tested positive for diarrhoeagenic E. coli. Although surface water may contain a wide variety of pathogens from both natural and human sources, E. coli was among a short list of pathogens that are predicted to be responsible for over 97% of non-foodborne illnesses, according to the CDC and the World Health Organization (WHO). E. coli alongside Salmonella enterica, Campylobacter jejuni, Giardia lamblia, and Cryptosporidium spp. were considered “reference pathogens” representative of the fate and transport of other waterborne pathogens of concern and exhibited corresponding dose-response relationships. E. coli was suggested and tested as proxies for other waterborne diseases including Hepatitis A, Salmonella typhi infection, Salmonellosis, and Streptococcus pneumonia invasive disease. In addition to pathogens, E. coli concentrations and survival in the aquatic environment also showed a positive correlation with nutrient levels such as ammonia-nitrogen, dissolved organic carbon, and phosphorus. Depending on the type, E. coli infection can cause a variety of human illnesses from mild diarrhea to acute hemorrhagic colitis. Despite the potential health risks, underserved communities and vulnerable groups were often disproportionately exposed to E. coli contaminations and waterborne diseases.

Many studies reported environmental disparities in minorities and underserved communities. For example, higher exposure to concentrated animal feeding operations (CAFOs), the main nonpoint pollution sources of pathogens, have been reported among less educated and low-income communities in North Carolina, black and Hispanic populations in Ohio, low-income, and people of color communities in Maryland, and black communities in Mississippi. High levels of mercury have been detected in surface water in Florida, especially in neighborhoods with a high percentage of people of color and low-income communities. Low-income and minorities in Philadelphia are disproportionally residing near waterways impacted by combined sewer overflows, and they are expected to experience higher potential health risks including a higher rate of waterborne diseases. Underserved communities are more likely to experience leachate from landfills, which can serve as a source of enteric pathogens. Studies have shown that underserved communities receive the discriminatory and unfair distribution of local environmental health policies and land planning which impact their access to safe drinking water supplies. Health insurance is less prevalent in underserved communities which can impact their responses to pathogens and waterborne diseases. Nevertheless, research lacks an adequate and critical investigation of the impacts of environmental justice metrics on waterborne disease prevalence and how that can be considered in cultivating strategies to mitigate pathogen transfer.

Exposure to contaminated water bodies or adverse impacts from exposure is more prevalent in certain groups. Waterborne disease rates and mortality are higher in children, mainly due to underdeveloped immune systems and a higher chance of exposure. The highest number of waterborne diseases in Florida occurred among children under 5 and elderly over 74 when compared to other age groups. The homeless population is also at greater risk of exposure, mainly due to the use of contaminated surface water for bathing or washing their belongings. Additionally, other vulnerable populations are at higher risk of adverse health impacts upon exposure including pregnant women (who can transfer pathogens to the fetus in utero or shortly after delivery) and immunocompromised populations such as transparent recipients, chemotherapy patients, and HIV positive individuals. Despite the potential for vulnerable groups to suffer worse health consequences from waterborne diseases, research on the likelihood of certain populations' exposure to pathogen-contaminated water is lacking. These data highlighted the challenges posed by waterborne diseases in underserved communities with the special attention to the vulnerable groups and the need for public health strategies and water management practices to mitigate the threat of waterborne diseases.

Stormwater best management practices (BMPs) are the main methods to control stormwater runoff and reduce its negative impacts by controlling flooding and peak runoff rate, managing runoff volume, improving water quality, and reducing erosion. Different types of BMPs include point BMPs (e.g., constructed wetland, bioretention, etc.), linear BMPs (e.g., grassed swale, infiltration trench, etc.), and area BMPs (e.g., green roof, porous pavement, etc.). Stormwater BMPs can improve water quality by reducing contaminant loadings. For example, BMPs have been effective in nutrient removal such as nitrogen and phosphorus. In addition, E. coli can be reduced by BMPs, such as retention ponds, wetland basins, and bioretention, in a few different ways including capturing and filtering the stormwater runoff. For example, wetland basin, retention pond, and detention basin reduced E. coli. in the effluent by 86%, 83%, and 44%. Stormwater BMPs can benefit communities on social, economic, and health levels, especially underserved communities, by improving urban aesthetics, promoting public health, and creating jobs. Although BMPs can be an effective tool for managing stormwater quality and reducing the risk of waterborne disease, there is little to no study that investigated the extent to which BMPs can reduce waterborne disease quantitatively, neither experimentally nor through modeling, especially at a larger scale (e.g., state level).

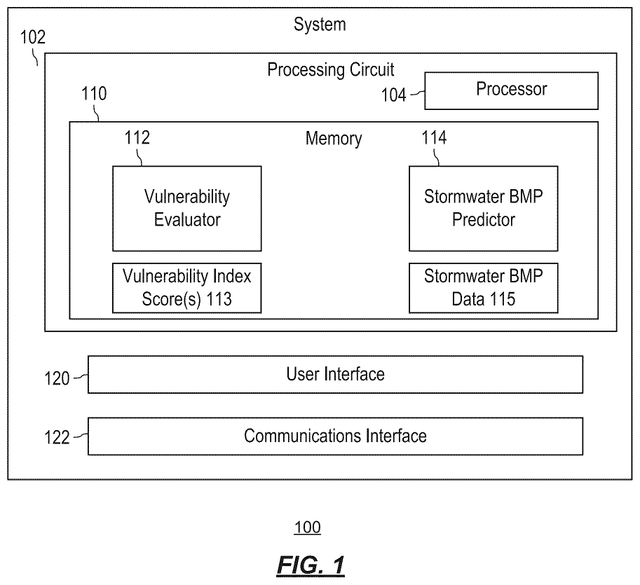

Referring to FIG. 1, a system 100 for determining an area's vulnerability to waterborne diseases and evaluating stormwater BMPs to mitigate vulnerabilities is shown. According to some implementations, the system 100 is shown to include a processing circuit 102 that includes a processor 104 and a memory 110. The processor 104 can be a general-purpose processor, an ASIC, one or more FPGAs, a group of processing components, or other suitable electronic processing structures. In some embodiments, the processor 104 is configured to execute program code stored on the memory 110 to cause the system 100 to perform one or more operations, as described below in greater detail. It will be appreciated that, in embodiments where the system 100 is part of another computing device, the components of the system 100 may be shared with, or the same as, the host device. For example, if the system 100 is implemented via a server (e.g., a cloud server), then the system 100 may utilize the processing circuit, processor(s), and/or memory of the server to perform the functions described herein.

The memory 110 can include one or more devices (e.g., memory units, memory devices, storage devices, etc.) for storing data and/or computer code for completing and/or facilitating the various processes described in the present disclosure. In some embodiments, the memory 110 includes tangible (e.g., non-transitory), computer-readable media that stores code or instructions executable by the processor 104. Tangible, computer-readable media refers to any physical media that is capable of providing data that causes the system 100 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, the memory 110 can include RAM, ROM, hard drive storage, temporary storage, non-volatile memory, flash memory, optical memory, or any other suitable memory for storing software objects and/or computer instructions. The memory 110 can include database components, object code components, script components, or any other type of information structure for supporting the various activities and information structures described in the present disclosure. The memory 110 can be communicably connected to the processor 104, such as via the processing circuit 102, and can include computer code for executing (e.g., by the processor 104) one or more processes described herein.

While shown as individual components, it will be appreciated that the processor 104 and/or the memory 110 can be implemented using a variety of different types and quantities of processors and memory. For example, the processor 104 may represent a single processing device or multiple processing devices. Similarly, the memory 110 may represent a single memory device or multiple memory devices. Additionally, in some embodiments, the system 100 may be implemented within a single computing device (e.g., one server, one housing, etc.). In other embodiments, the system 100 may be distributed across multiple servers or computers (e.g., that can exist in distributed locations). For example, the system 100 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. For example, virtualization software may be employed by the system 100 to provide the functionality of a number of servers that is not directly bound to the number of computers in the system 100.

The memory 110 is shown to include a vulnerability evaluator 112 configured to determine an area's (e.g., geographical area) vulnerability to waterborne diseases. Specifically, the vulnerability evaluator 112 is configured to generate a vulnerability index score 113 based on a set of metrics that includes socioeconomic data and historical rates of waterborne diseases for the area. The “vulnerability index score,” as described herein, is a quantitative metric that is generally indicative of an area's vulnerability to waterborne diseases. In some implementations, the vulnerability evaluator 112 includes a multi-variate model that outputs the vulnerability index score 113 based on input data (e.g., variables). In some implementations, the vulnerability evaluator 112 includes a predictive model that generates the vulnerability index score 113. In some such implementations, the predictive model is a machine learning model (e.g., a convolutional neural network (CNN), an artificial neural network (ANN), or the like); however, other types of predictive models are contemplated herein.

Generally, the vulnerability evaluator 112 may obtain input data—which, as mentioned above, includes socioeconomic data and historical rates of waterborne diseases for the geographical area—in the form of one or more data sets. In some implementations, the socioeconomic data and historical waterborne disease data are received from a remote computing device (e.g., an external server or other computer). For example, the socioeconomic data and historical waterborne disease data may be transmitted from a remote computer to the system 100, e.g., which is implemented via a cloud server, for processing. In some implementations, the socioeconomic data and the historical waterborne disease data are obtained via a user input. For example, a user may manually upload datasets to system 100 and/or may input data via a user interface. In some implementations, the socioeconomic data and the historical waterborne disease data are retrieved from a database, which may be internal to or remote from the system 100. Depending on the embodiment, a geographical area of interest may refer to a county, country, state, town, zip-code, municipality or any type of defined geographical area where sufficient socioeconomic data and historical waterborne disease data is available.

As described herein, “socioeconomic data” generally includes a plurality of data points relating to social and economic conditions in the geographical area of interest. For example, socioeconomic data can include, but is not limited to, race and ethnicity data (e.g., percentages of the population in the area that identify as non-white, Hispanic, etc.), employment data (e.g., percent of the population in the area that is unemployed), income data (e.g., percent of the population in the area that is below the poverty line, household income, etc.), health insurance data (e.g., percent of the population in the area that does not have health insurance), and the like. It should be appreciated that various other socioeconomic data points may be used and are contemplated herein, even if not expressly recited.

In some embodiments, the vulnerability evaluator 112 may calculate a vulnerability index score 113 for a geographical area by considering a variety of metrics that are derived or based on the input data or data points described above. In particular, the metrics may include including environmental justice (“EJ”) metrics for at risk populations as well as the historic waterborne disease metrics. For a given geographical area, the vulnerability evaluator 112 may first standardize each metric. For metrics that are expected to have a direct relationship with the vulnerability, such as % Hispanic, % educational attainment, etc., the vulnerability evaluator 112 may standardize the metrics using Eq (1). Other methods for standardizing metrics may be used.

$$Standardized Metric = \frac{{S}_{i} - {S}_{min}}{{S}_{max} - {S}_{min}} \qquad Eq ( 1 )$$

For metrics that are expected to have an inverse relation to vulnerability, such as the median of household income, etc., the vulnerability evaluator 112 may standardize the metrics using Eq (2). Other methods for standardizing metrics may be used.

$$Standardized Metric = \frac{{S}_{max} - {S}_{i}}{{S}_{max} - {S}_{min}} \qquad Eq ( 2 )$$

Where Sᵢ is the observed value for each metric at a given county, Sₘᵢₙ and Sₘₐₓ are the minimum and maximum values of the metric across all counties, respectively.

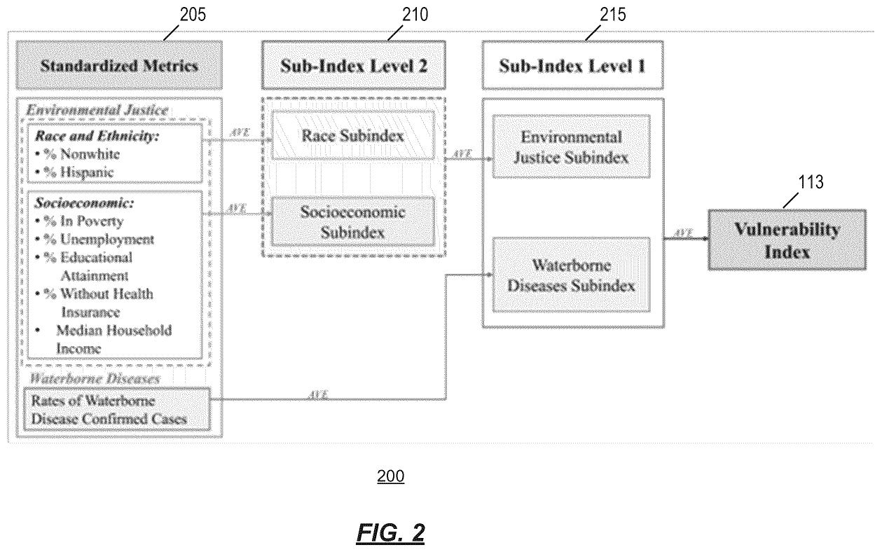

FIG. 2 is an illustration of one example of how the vulnerability evaluator 112 may calculate the vulnerability index score for a geographical area. As shown, the vulnerability evaluator 112 starts by collecting various metrics including environmental justice metrics and waterborne disease metrics. The vulnerability evaluator 112 may then standardize the metrics as described above to create the standardized metrics 205. These standardized metrics 205 may then be used to create the vulnerability index scores 113.

In the example shown, the vulnerability evaluator 112 may further drill down the standardized metrics 205 to create what are referred to as the sub-indexes. These sub-indexes may then be separately weighted and combined to form the vulnerability index scores 113. Example sub-indexes are shown in the sub-indexes 210 and 215 of FIG. 2. In the example shown, the race and ethnicity related environmental justice metrics and socioeconomic related environmental justice metrics are used to create a race sub-index and a socioeconomic sub-index. These sub-indexes are then combined (using selected weights) to form the environmental justice sub-index. Finally, the environmental justice sub-index is combined with a waterborne diseases sub-index to form the vulnerability index scores 113. Note the use of sub-indexes allows the vulnerability evaluator 112 to change or adjust how particular groups of metrics affect the generated vulnerability index scores 113 by adjusting one or more weights.

Returning to FIG. 1, the memory 110 is also shown to include a stormwater best management practices (BMP) predictor 114 configured to evaluate the impact of various stormwater BMPs on the vulnerability index for a given area. In particular, the stormwater BMP predictor 114 can simulate the effect of various stormwater BMPs on the geographical area of interest to determine how the stormwater BMPs affect the vulnerability index score 113 for the area (e.g., by increasing or decreasing the score). Generally, the stormwater BMP predictor 114 may evaluate the vulnerability index score 113 of the area with respect to a predefined set of stormwater BMPs.

For example, the set of stormwater BMPs may include, but are not limited to, grass strips, bioretention, catch basis insert, composite, detention basins, green roofs, hydrodynamic separation devices, high-rate media filtration, media filters, oil and grit separation and baffle boxes, porous pavements, retention ponds, wetland basins, grass swales, and uncategorized manufactured devices, and wetland channels. Other types of stormwater BMPs may be supported.

In some implementations, the stormwater BMP predictor 114 simulates the effect of stormwater BMPs on the vulnerability index score 113 for a geographical area using stormwater BMP data 115. The stormwater BMP data 115 may include historical data collected about the effectiveness of various BMPs. For example, the stormwater BMP predictor 114 may collect BMP data 115 from one or more external sources such as International Stormwater BMP databases. For a metric such as waterborne diseases, the BMP data 115 may indicate the measured levels of certain waterborne diseases before and after the implementation of each BMP.

In some embodiments, the stormwater BMP predictor 114 may simulate the impact of a stormwater BMP on the vulnerability index score 113 of a selected geographical area using the BMP data 115 for the stormwater BMP. The stormwater BMP predictor 114 may use the BMP data 115 to estimate the change to the historical waterborne disease data (or other stormwater related metric) used to calculate the vulnerability index score 113. The stormwater BMP predictor 114 may then recalculate the vulnerability index score 113 using the changed stormwater related metric. The difference between the original vulnerability index score 113 and the recalculated vulnerability index score 113 based on the stormwater BMP may be presented as the simulated impact of the stormwater BMP.

In some embodiments, the stormwater BMP predictor 114 may simulate the impact of a stormwater BMP on the vulnerability index score 113 of a geographical area using a machine learning model. For example, the machine learning model may be trained using historical data from various different areas, which includes respective waterborne disease rates and stormwater BMPs that have been implemented in the corresponding areas. The stormwater BMP predictor 114 may then output an indication of how one or more different stormwater BMPs will impact the vulnerability index score 113 for the geographical area.

The system 100 is shown to include a user interface 120, in some implementations. Generally, the user interface 120 includes one or more components that allow a user to interact with system 100, e.g., by entering and/or viewing data. In some implementations, user interface 120 includes a display, such as an LCD or LED display, on which data and/or graphics can be displayed. For example, the user interface 120 can include a display that is capable of presenting graphs, text, and other graphical elements to a user, e.g., to present the results of the vulnerability and stormwater BMP evaluations described herein. In some implementations, the user interface 120 includes one or more user input devices that allow a user to enter data, including but not limited to, a keyboard, a mouse, a number pad, a keypad, and the like. In some implementations, the user interface 120 includes a touchscreen that can both display GUIs and receive user inputs. Other suitable types of user interface components are contemplated herein.

The system 100 is also shown to include a communications interface 122 that facilitates communications between the system 100 and any external components or devices. For example, the communications interface 122 can provide means for transmitting data to, or receiving data from, remote computing devices (e.g., laptops/desktops, smartphones, smart watches, servers, tablets, etc.). Accordingly, the communications interface 122 can be or can include a wired or wireless communications interface (e.g., jacks, antennas, transmitters, receivers, transceivers, wire terminals, etc.) for conducting data communications, or a combination of wired and wireless communication interfaces. In some embodiments, communications via the communications interface 122 are direct (e.g., local wired or wireless communications) or via a network (e.g., a WAN, the Internet, a cellular network, etc.). For example, the communications interface 122 may include one or more Ethernet ports for communicably coupling the system 100 to a network (e.g., the Internet). In another example, the communications interface 122 can include a Wi-Fi transceiver for communicating via a wireless communications network. In yet another example, the communications interface 122 may include cellular or mobile phone communications transceivers.

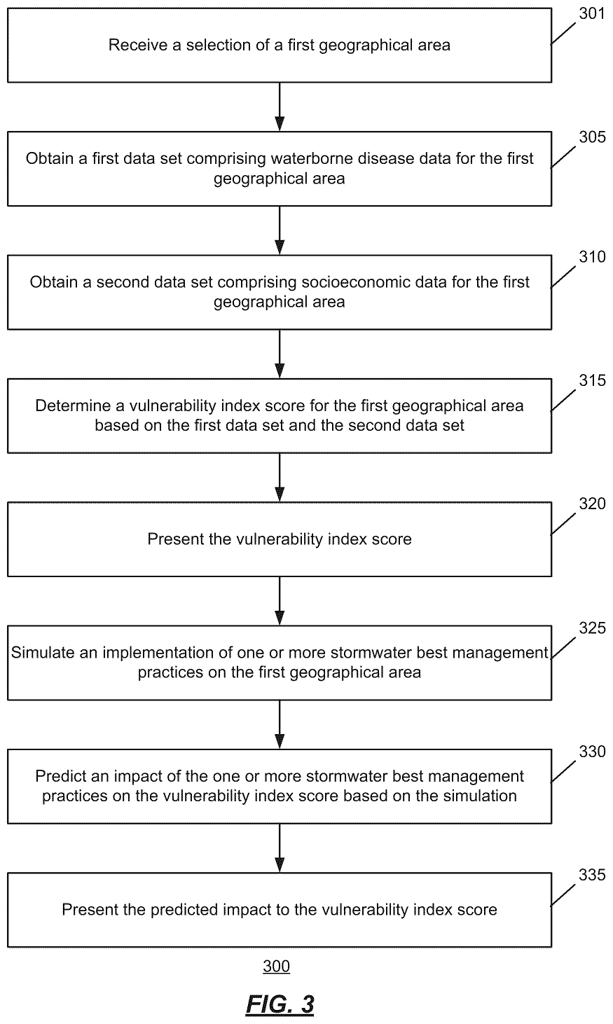

FIG. 3 is an illustration of a method 300 for predicting an impact of one or more stormwater BMPs on a vulnerability index score for a geographical area. The method 300 may be implemented by the system 100.

At 301, a selection of a first geographical area is received. The selection may be received by the system 100 through a user interface 120. The first geographical area may be a geographical area of interest that a user is interested in determining how one or more stormwater BMPs would affect a vulnerability index score 113 associated with the geographical area of interest. The first geographical area may correspond to a defined geographical area such as a town, city, county, state, country, or zip code. Depending on the embodiment, the user may select the geographical area of interest by providing a name, zip code, or other identifier that corresponds to the geographical area. Alternatively, the user may select the first geographical area by selecting the area on a map displayed in the user interface 120. Other methods for selecting a geographical area may be used.

At 305, a data set comprising waterborne disease data for the first geographical area is obtained. The waterborne disease data may be obtained by the vulnerability evaluator 112. The waterborne disease data may be historical waterborne disease data for the selected geographical area and may include metrics such as rates of infection due to certain waterborne diseases recorded for the geographical area. The data may be retrieved from one or more public or private sources of waterborne disease data.

At 310, a data set comprising socioeconomic data for the first geographical area is obtained. The socioeconomic data may be obtained by the vulnerability evaluator 112. The socioeconomic data may include metrics related to ethnicity, employment, health, age, and education. The data may be retrieved from one or more public or private sources of socioeconomic data.

At 315, a vulnerability index score for the first geographical area is determined. The vulnerability index score 113 may be determined by the vulnerability evaluator 112 using metrics from the socioeconomic data and the waterborne disease data. Depending on the embodiment, the metrics may be standardized as described above. When calculating the vulnerability index score 113, the user may adjust weights associated with each metric to control how the value of each metric affects the vulnerability index score 113. In some embodiments, rather than calculate the vulnerability index score 113 for the selected first geographical area, the vulnerability index scores 113 for a set of geographical areas may have been precomputed and stored by the vulnerability evaluator 112. Alternatively or additionally, the vulnerability evaluator 112 may download the precomputed vulnerability index scores 113 from another computer or remote server.

At 320, the vulnerability index score is presented. The vulnerability index score 113 may be presented to the user by the vulnerability evaluator 112. The score 113 may be presented in the same or different user interface 120 that was used to select the first geographical area.

At 325, the implementation of one or more stormwater BMPs on the vulnerability index score 113 are simulated. The one or more stormwater BMPs may be simulated by the stormwater BMP predictor 114 using stormwater BMP data 115. The stormwater BMP data 115 may be data collected from a variety of locations regarding the effects of the BMPs on certain waterborne disease metrics. In some embodiments, the stormwater BMP predictor 114 may simulate the implementation of the stormwater BMP on the vulnerability index score 113 by determining the expected changes to one or more waterborne disease metrics based on the stormwater BMP data 115. For example, if the stormwater BMP data 115 showed that a particular stormwater BMP reduced a certain disease by 5% on average after implementation, the stormwater BMP predictor 114 may simulate the implementation of the particular disease by adjust ting the metric associated with the disease that was used in the vulnerability index score by 5%. Alternatively or additionally, a machine learning model may predict the changes to one or more of the waterborne disease metrics based on the BMP data 115.

In some embodiments, the stormwater BMP predictor 114 may simulate each stormwater BMP reflected in the BMP data 115. Alternatively, the user may select the one or more stormwater BMPs that they are interested in, and the stormwater BMP predictor 114 may simulate the selected one or more stormwater BMPs.

At 330, the impact of the one or more stormwater BMPs on the vulnerability index score is predicted. The impact may be predicted by the vulnerability evaluator 112. In some embodiments, the vulnerability evaluator 112 may predict the impact of a stormwater BMP based on the simulation. In particular, the vulnerability evaluator 112 may predict the impact of a stormwater BMP 113 by recalculating the vulnerability index score 113 using the updated values of certain waterborne disease metrics as predicted by the simulation for the stormwater BMP at 325. The vulnerability evaluator 112 may recalculate the vulnerability index score 113 for each of the one or more stormwater BMPs.

At 335, the predicted impact to the vulnerability index scores is presented. The predicted impact to the vulnerability index score 113 for each of the one or more stormwater BMPs may be presented by the vulnerability evaluator 112. The predicted impact may be presented using the same or different user interface 120 that was used to select the first geographical area. In some embodiments, the predicted impact may be presented by displaying each of the one or more stormwater BMPs along with the percent or amount of change that is predicted for each stormwater BMP.

The construction and arrangement of the systems and methods as shown in the various implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A computer implemented method for determining a vulnerability index score comprising:
obtaining, by one or more computing devices, a first data set comprising waterborne disease case data for a first geographical area;
obtaining, by the one or more computing devices, a second data set comprising socioeconomic data for the first geographical area;
determining, by the one or more computing devices, the vulnerability index score for the first geographical area based on the first data set and the second data set; and
presenting, by the one or more computing devices, a first graphical user interface that indicates the vulnerability index score.

2. The method of claim 1, further comprising:
simulating, by the one or more computing devices, an implementation of one or more stormwater best management practices (BMPs) on the first geographical area; and
predicting, by the one or more computing devices, an impact on the vulnerability index score due to the one or more stormwater BMPs based on the simulation.

3. The method of claim 2, wherein the impact of the one or more stormwater BMPs on the vulnerability index score is simulated using a machine learning model.

4. The method of claim 2, further comprising:
presenting, by the one or more computing devices, a second graphical interface that indicates the one or more stormwater BMPs and the predicted impact on the vulnerability index score due to the one or more stormwater BMPs.

5. The method of claim 2, further comprising:
updating, by the one or more computing devices, the first graphical interface to include an indication of the one or more stormwater BMPs and the predicted impact on the vulnerability index score due to the one or more stormwater BMPs.

6. The method of claim 1, wherein the first geographical area is a country, a state, a county, or a city.

7. The method of claim 1, wherein the vulnerability index score is determined using a predictive model.

8. The method of claim 7, wherein the predictive model is a machine learning model.

9. The method of claim 1, wherein the first graphical user interface includes a map of at least the first geographical area.

10. The method of claim 9, wherein the map visually indicates the vulnerability index score.

11. A system for determining a vulnerability index score comprising:
at least one computing device;
a computer-readable medium with computer-executable instructions stored thereon that when executed by the at least one computing device cause the at least one computing device to:
obtain a first data set comprising waterborne disease case data for a first geographical area;
obtain a second data set comprising socioeconomic data for the first geographical area;
determine the vulnerability index score for the first geographical area based on the first data set and the second data set; and
present a first graphical user interface that indicates the vulnerability index score.

12. The system of claim 11, further comprising computer-executable instructions stored thereon that when executed by the at least one computing device cause the at least one computing device to:
simulate an implementation of one or more stormwater best management practices (BMPs) on the first geographical area; and
predict an impact on the vulnerability index score due to the one or more stormwater BMPs based on the simulation.

13. The system of claim 12, wherein the impact of the one or more stormwater BMPs on the vulnerability index score is simulated using a machine learning model.

14. The system of claim 12, further comprising computer-executable instructions stored thereon that when executed by the at least one computing device cause the at least one computing device to:
present a second graphical interface that indicates the one or more stormwater BMPs and the predicted impact on the vulnerability index score due to the one or more stormwater BMPs.

15. The system of claim 12, further comprising computer-executable instructions stored thereon that when executed by the at least one computing device cause the at least one computing device to:
update the first graphical interface to include an indication of the one or more stormwater BMPs and the predicted impact on the vulnerability index score due to the one or more stormwater BMPs.

16. The system of claim 11, wherein the first geographical area is a country, a state, a county, or a city.

17. The system of claim 11, wherein the vulnerability index score is determined using a predictive model.

18. The system of claim 17, wherein the predictive model is a machine learning model.

19. The system of claim 11, wherein the first graphical user interface includes a map of at least the first geographical area.

20. The system of claim 19, wherein the map visually indicates the vulnerability index score.
